(19) United States
(12) Patent Application Publication
(10) Pub. No.: US 2022/0009879 A1
(43) Pub. Date: Jan. 13, 2022

(54) BRANCHED AMINO ACID SURFACTANTS

(71) Applicant: AdvanSix Resins & Chemicals LLC, Parsippany, NJ (US)

(72) Inventors: Edward Asirvatham, Chatham, NJ (US); Andrei Honciuc, Iasi (RO); Voichita Mihali, Basel (CH)

(21) Appl. No.: 17/365,775

(22) Filed: Jul. 1, 2021

Related U.S. Application Data
(60) Provisional application No. 63/049,744, filed on Jul. 9, 2020.

(51) Int. Cl. C07C 229/12

(52) U.S. Cl. CPC C07C 229/12; B01F 17/0042

(57) ABSTRACT

The present disclosure provides derivatives of amino acids that have branched alkyl structures and surface-active properties. The amino acid can be naturally-occurring or synthetic, or they may be obtained via a ring-opening reaction of a lactam, such as caprolactam. The amino acid may be functionalized to form a compound that is surface-active and have advantageous surfactant characteristics. The compounds of the present disclosure have low critical micelle concentrations (CMC) as well as superior ability to lower the surface tension of a liquid.

### CROSS-REFERENCE TO RELATED APPLICATION

This application claims priority to U.S. Provisional Application No. 63/049,744, filed Jul. 9, 2020, which is herein incorporated by reference in its entirety.

### FIELD

The present disclosure pertains to derivatives of amino acids and methods for their synthesis, wherein the amino acid derivatives include branched alkyl structures and have surface-active properties.

### BACKGROUND

Surfactants (molecules with surface-active properties) are an important class of molecules with highly sought-after characteristics. Surfactants may be uncharged, zwitterionic, cationic, or anionic. Often, these compounds are amphiphilic molecules with a water-insoluble hydrophobic “tail” group and a water-soluble hydrophilic “head” group. These compounds may adsorb at an interface, such as an interface between two liquids, a liquid and a gas, or a liquid and a solid. In the case of an interface between water and oil, the hydrophilic head group extends into the water, while the hydrophobic tail extends into the oil. When added to water, the hydrophilic head group extends into the water, while the hydrophobic tail extends into the air. The presence of the surfactant disrupts the intermolecular interaction between water molecules, replacing it with weaker interactions between water molecules and the surfactant. This results in lowered surface tension and can also serve to stabilize the interface.

At sufficiently high concentrations, surfactants may form aggregates to limit the exposure of the hydrophobic tail to the polar solvent. One such aggregate is a micelle, in which the molecules are arranged in a sphere with the hydrophobic tails inside the sphere and the hydrophilic heads on the outside to interact with a polar solvent. The effect that a given compound has on surface tension and the concentration at which it forms micelles may serve as defining characteristics for a surfactant.

Surfactants are widely used in commercial applications in formulations ranging from detergents to hair care products to cosmetics. Compounds with surface-active properties are used as soaps, detergents, lubricants, wetting agents, foaming agents, and spreading agents, among others. Thus, there is an ongoing need to identify and synthesize such compounds.

However, solely from its structure, it may be difficult to predict whether a given compound would have surface-active properties, let alone other important characteristics such as interfacial adsorption dynamics, minimum surface tension achievable, and/or ability to wet hydrophobic and/or oleophobic surfaces, which are also integral to whether the compound would become a useful surfactant. Certain amino acids and their derivatives, for example, are desirable as building blocks for surfactants, but the selection of which amino acids to use is far from intuitive. Synthesis of such compounds adds another layer of difficulty due to the differences of solubilities attributable to different elements and moieties present in the same molecules. There remains a need for high-efficacy surfactants that can be readily synthesized at commercial scale via straightforward routes.

### SUMMARY

The present disclosure provides derivatives of amino acids that have a branched alkyl structure, and which exhibit surface-active properties. The amino acids may be naturally occurring or synthetic amino acids, or they may be obtained via ring-opening reactions of molecules such as lactams, for example caprolactam. The amino acids may be functionalized to form compounds with surface-active properties. Characteristically, these compounds may have low critical micelle concentrations (CMC) and/or the ability to reduce the surface tension of a liquid.

The present disclosure provides compounds of Formula I, below, also referred to herein as the surfactant:

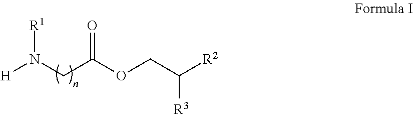

wherein R¹ is chosen from hydrogen, an oxygen atom, and C₁-C₆ alkyl, wherein the C₁-C₆ alkyl may be substituted with carboxylates, hydroxyls, sulfonyls, or sulfonates; n is an integer from 2 to 5 (including 2 and 5); R² is C₅-C₁₂ alkyl; R³ is C₃-C₁₀ alkyl; the terminal nitrogen is optionally further substituted with R⁴, wherein R⁴ is chosen from hydrogen, an oxygen atom, and C₁-C₆ alkyl, wherein the C₁-C₆ alkyl may be substituted with carboxylates, hydroxyls, sulfonyls, or sulfonates; and an optional counterion may be associated with the compound and, if present, the counterion may be 4-methylbenzenesulfonate.

One specific compound provided by the present disclosure is 6-((2-butyloctyl)oxy)-6-oxohexan-1-aminium 4-methylbenzenesulfonate, having the following formula:

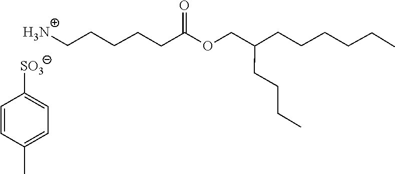

The above mentioned and other features of the disclosure, and the manner of attaining them, will become more apparent and will be better understood by reference to the following description of embodiments taken in conjunction with the accompanying drawings.

### BRIEF DESCRIPTION OF THE DRAWINGS

FIG. 1 shows a plot of surface tension versus concentration measured at pH=7 as described in Example 2, wherein the Y axis depicts the surface tension (γ) in millinewtons per meter (mN/m) and the X axis depicts the concentration (c) in millimoles (mM).

FIG. 2 shows a plot of dynamic surface tension as change in surface tension versus time as described in Example 3, wherein the Y axis depicts the surface tension in millinewtons per meter (mN/m) and the X axis depicts the surface age in milliseconds (ms).

### DETAILED DESCRIPTION

### I. Definitions

As used herein, the phrase “within any range defined between any two of the foregoing values” literally means that any range may be selected from any two of the values listed prior to such phrase regardless of whether the values are in the lower part of the listing or in the higher part of the listing. For example, a pair of values may be selected from two lower values, two higher values, or a lower value and a higher value.

As used herein, the word “alkyl” means any saturated carbon chain, which may be a straight or branched chain.

As used herein, the phrase “surface-active” means that the associated compound is able to lower the surface tension of the medium in which it is dissolved, and/or the interfacial tension with other phases, and, accordingly, may be adsorbed at the liquid/vapor and/or other interfaces. The term “surfactant” may be applied to such a compound.

With respect terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error or minor adjustments made to optimize performance, for example. In the event it is determined that individuals having ordinary skill in the relevant arts would not readily ascertain values for such reasonably small differences, the terms “about” and “approximately” can be understood to mean plus or minus 10% of the stated value.

### II. Surfactant Formulas

The present disclosure provides derivatives of amino acids having a branched alkyl structure. The amino acids may be naturally occurring or synthetic, or they may be obtained from ring-opening reactions of lactams, such as caprolactam. The compounds of the present disclosure have been shown to have surface-active properties, and may be used as surfactants and wetting agents, for example. In particular, the present disclosure provides compounds of Formula I, shown below the surfactant:

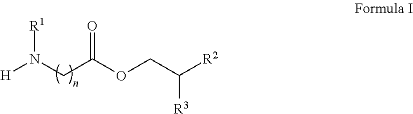

wherein R¹ is chosen from hydrogen, an oxygen atom, and C₁-C₆ alkyl, wherein the C₁-C₆ alkyl may be substituted with carboxylates, hydroxyls, sulfonyls, or sulfonates; n is an integer from 2 to 5 (including 2 and 5); R² is C₅-C₁₂ alkyl; R³ is C₃-C₁₀ alkyl; the terminal nitrogen is optionally further substituted with R⁴, wherein R⁴ is chosen from hydrogen, an oxygen atom, and C₁-C₆ alkyl, wherein the C₁-C₆ alkyl may be substituted with carboxylates, hydroxyls, sulfonyls, or sulfonates; and an optional counterion may be associated with the compound and, if present, the counterion may be 4-methylbenzenesulfonate.

One specific compound provided by the present disclosure is One specific compound provided by the present disclosure is 6-((2-butyloctyl)oxy)-6-oxohexan-1-aminium 4-methylbenzenesulfonate, having the following formula:

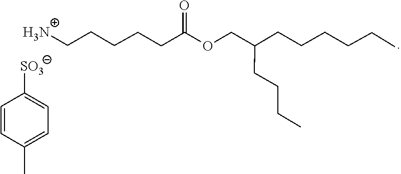

### III. Synthesis

The present surfactant compounds may be synthesized by various methods. One such method includes opening a lactam to yield an amino acid having an N-terminus and a C-terminus. The C-terminus may then react with an alcohol under acidic conditions to provide an amino acid ester. The N-terminus of the amino acid may react with an acid to yield an ammonium salt.

The amino acid may be naturally occurring or synthetic or may be derived from a ring opening reaction of a lactam, such as propiolactam, butyrolactam, valerolactam, and caprolactam, for example. The ring-opening reaction may be either an acid or alkali catalyzed reaction, and an example of an acid catalyzed reaction is shown below in Scheme 1 in connection with caprolactam.

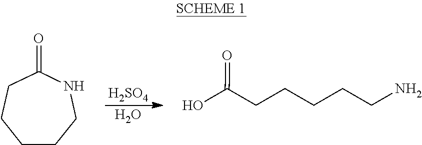

The amino acid may have as few as 2 or as many as 5 carbons between the N- and C-termini. The alkyl chain may be branched or straight. The alkyl chain may be interrupted with nitrogen, oxygen, or sulfur. The alkyl chain may be further substituted with one or more substituents selected from the group consisting of hydroxyl, amino, amido, sulfonyl, sulfonate, carboxyl, and carboxylate. For example, the amino acid may be 6-aminohexanoic acid.

The amino acid may be further elaborated as shown below in Scheme 2, wherein R² may be C₅-C₁₂ alkyl, and R³ may be C₃-C₁₀ alkyl. For example, the amino acid may undergo an esterification reaction in which the amino acid is treated with an alcohol under acidic conditions to provide an amino acid ester.

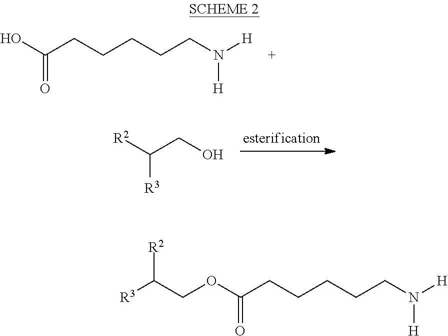

The resultant amino acid ester may then be protonated to give an ammonium salt, as shown below in Scheme 3. Protonation may be accomplished by treating the amino acid ester with an acid, such as para-toluenesulfonic acid. The identity of the acid determines the counterion present in the salt. For example, treatment with para-toluenesulfonic acid results in the 4-methylbenzene sulfonate salt of the ammonium species.

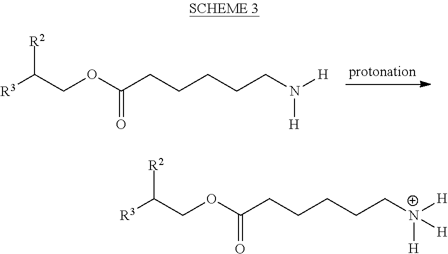

### IV. Surfactant Properties

The compounds of the present disclosure demonstrate surface-active properties. These properties may be measured and described by various methods. One method by which surfactants may be described is by the molecule's critical micelle concentration (CMC). CMC may be defined as the concentration of a surfactant at which micelles form, and above which all additional surfactant is incorporated into micelles.

As surfactant concentration increases, surface tension decreases. Once the surface is completely overlaid with surfactant molecules, micelles begin to form. This point represents the CMC, as well as the minimum surface tension. Further addition of surfactant will not further affect the surface tension. CMC may therefore be measured by observing the change in surface tension as a function of surfactant concentration. One such method for measuring this value is the Wilhemy plate method. A Wilhelmy plate is usually a thin iridium-platinum plate attached to a balance by a wire and placed perpendicularly to the air-liquid interface. The balance is used to measure the force exerted on the plate by wetting. This value is then used to calculate the surface tension (γ) according to Equation 1:

γ=F/I cos θ Equation 1:

wherein I is equal to the wetted perimeter (2w+2d, in which w and d are the plate thickness and width, respectively) and cos θ, the contact angle between the liquid and the plate, is assumed to be 0 in the absence of an extant literature value.

Another parameter used to assess the performance of surfactants is dynamic surface tension. The dynamic surface tension is the value of the surface tension for a particular surface or interface age. In the case of liquids with added surfactants, this can differ from the equilibrium value. Immediately after a surface is produced, the surface tension is equal to that of the pure liquid. As described above, surfactants reduce surface tension; therefore, the surface tension drops until an equilibrium value is reached. The time required for equilibrium to be reached depends on the diffusion rate and the adsorption rate of the surfactant.

One method by which dynamic surface tension is measured relies upon a bubble pressure tensiometer. This device measures the maximum internal pressure of a gas bubble that is formed in a liquid by means of a capillary. The measured value corresponds to the surface tension at a certain surface age, the time from the start of the bubble formation to the occurrence of the pressure maximum. The dependence of surface tension on surface age can be measured by varying the speed at which bubbles are produced.

Surface-active compounds may also be assessed by their wetting ability on solid substrates as measured by the contact angle. When a liquid droplet comes in contact with a solid surface in a third medium, such as air, a three-phase line forms among the liquid, the gas and the solid. The angle between the surface tension unit vector, acting at the three-phase line and tangent at the liquid droplet, and the surface is described as the contact angle. The contact angle (also known as wetting angle) is a measure of the wettability of a solid by a liquid. In the case of complete wetting, the liquid is completely spread over the solid and the contact angle is 0°. Wetting properties are typically measured for a given compound at the concentration of 1-10×CMC, however, it is not a property that is concentration-dependent therefore measurements of wetting properties can be measured at concentrations that are higher or lower.

In one method, an optical contact angle goniometer may be used to measure the contact angle. This device uses a digital camera and software to extract the contact angle by analyzing the contour shape of a sessile droplet of liquid on a surface.

Potential applications for the surface-active compounds of the present disclosure include formulations for use as shampoos, hair conditioners, detergents, spot-free rinsing solutions, floor and carpet cleaners, cleaning agents for graffiti removal, wetting agents for crop protection, adjuvants for crop protection, and wetting agents for aerosol spray coatings.

It will be understood by one skilled in the art that small differences between compounds may lead to substantially different surfactant properties, such that different compounds may be used with different substrates, in different applications. For example, small changes in the hydrophobic portion of the surfactant, such as a difference in the number of carbons in an alkyl chain, the presence of a branched alkyl chain, the number of branches in a branched alkyl chain, and the number of carbons in each branch of a branched alkyl chain, may lead to different surfactant properties. Likewise, in the case of cationic and anionic surfactants, different counterions may substantively change the surfactant properties of a compound.

The compounds are effective as surface-active agents, useful for wetting or foaming agents, dispersants, emulsifiers, and detergents, among other applications.

The amount of the compounds disclosed herein used in a formulation may be as low as about 0.001 wt. %, about 0.05 wt. %, about 0.1 wt. %, about 0.5 wt. %, about 1 wt. %, about 2 wt. %, or about 5 wt. %, or as high as about 8 wt. %, about 10 wt. %, about 15 wt. %, about 20 wt. %, or about 25 wt. %, or within any range defined between any two of the foregoing values.

The following non-limiting Examples are provided to demonstrate the different properties of the different surfactants.

### EXAMPLES

Nuclear magnetic resonance (NMR) spectroscopy was performed on a Bruker 500 MHz spectrometer. The critical micelle concentration (CMC) was determined by the Wilhelmy plate method at 23° C. with a tensiometer (DCAT 11, DataPhysics Instruments GmbH) equipped with a Pt—Ir plate. Dynamic surface tension was determined with a bubble pressure tensiometer (Krüss BP100, Krüss GmbH), at 23° C. Contact angle was determined with the optical contact angle goniometer (OCA 15 Pro, DataPhysics GmbH) equipped with a digital camera.

### Example 1

### Synthesis of 6-((2-butyloctyl)oxy)-6-oxohexan-1-aminium 4-methylbenzenesulfonate

6-Aminohexanoic acid (38.11 mmol, 5 g) was dissolved in benzene (50 mL) in a 100 mL round bottom flask equipped with a Dean Stark trap. p-Toluenesulfonic acid monohydrate (38.11 mmol, 7.25 g) and 2-butyloctanol (38.11 mmol, 7.1 g, 8.5 mL) were added, and the mixture was heated to reflux for one week, until no further water was separated in the Dean Stark trap. The solvent was removed under vacuum and the product was crystallized from acetone at −20° C. to remove residual unreacted alcohol. The resultant white waxy solid was filtered to give 2-butyloctyl)oxy)-6-oxohexan-1-aminium 4-methylbenzenesulfonate in 82% yield. ¹H NMR (500 MHz, DMSO) δ 7.49 (d, J=8.0 Hz, 2H), 7.12 (dd, J=8.4, 0.6 Hz, 2H), 3.93 (d, J=5.7 Hz, 2H), 2.79-2.73 (m, 2H), 2.31-2.28 (m, 5H), 1.55-1.50 (m, 5H), 1.31-1.25 (m, 18H), 0.88-0.85 (m, 6H).

### Example 2

### Determination of Critical Micelle Concentration (CMC)

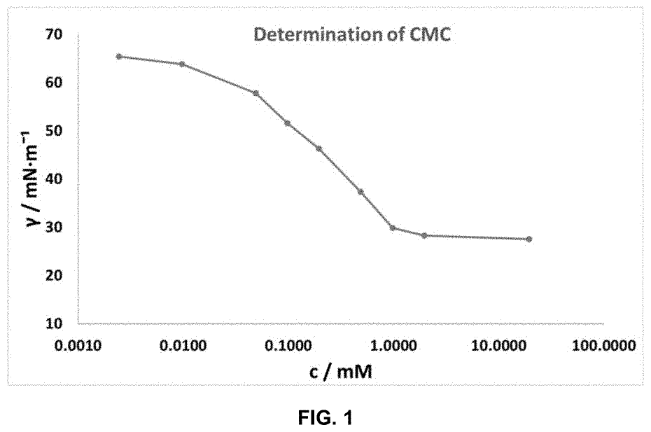

The critical micelle concentration (CMC) of the 6-((2-butyloctyl)oxy)-6-oxohexan-1-aminium 4-methylbenzenesulfonate from Example 1 was tested. From the change in surface tension with concentration in water, the CMC was determined to be about 2.12 mmol. The plateau value of minimum surface tension that can be reached by this surfactant is about 27 mN/m, namely 27 mN/m±3 mN/m. FIG. 1 is a plot of these results, showing surface tension versus. From the plot of the results, the surface tension at the CMC is equal to or less than about 30 mN/m, and the surface tension equal to or less than about 28.5 mN/m at a concentration of about 1.0 mmol or greater.

### Example 3

### Determination of Dynamic Surface Tension

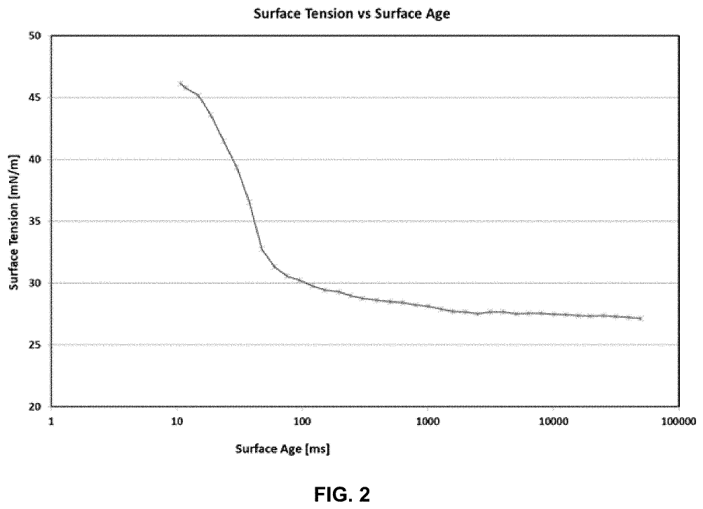

The dynamic surface tension of the 6-((2-butyloctyl)oxy)-6-oxohexan-1-aminium 4-methylbenzenesulfonate from Example 2 was determined with a bubble pressure tensiometer which measures the change of surface tension of a freshly created air-water interface with time. FIG. 2 presents a plot of the surface tension versus time, showing that surface tension in the time interval between 10 and 100 ms drops rapidly from about 46 mN/m to about 30 mN/m. In the time interval from 100 to 8,000 ms, the surface tension drops slowly from 30 mN/m to about 27 mN/m, approaching asymptotically the saturation value of the surface tension at the CMC.

### Example 4

### Determination of Wetting Properties

In addition to surface tension and surface dynamics, the wetting properties of the 6-((2-butyloctyl)oxy)-6-oxohexan-1-aminium 4-methylbenzenesulfonate from Example 3 were tested on various surfaces. For example, hydrophobic substrates such as polyethylene-HD exhibit surface wetting with a contact angle of 14.6°. On oleophobic and hydrophobic substrates such as Teflon, the measured contact angle was much less than that of water's contact angle of 119°, at 49.4° (Table 1).

TABLE 1

| | CA of | | CA of |
|---|---|---|---|
| Substrate | Surfactant (°) | Concentration | water (°) |
| Teflon | 49.4 | 10x CMC | 119 |
| Polyethylene-HD | 14.6 | 10x CMC | 93.6 |
| Nylon | 12.6 | 10x CMC | 50 |
| Polyethylene terephthalate | 13.2 | 10x CMC | 65.3 |

### Aspects

Aspect 1 is a compound of the following formula:

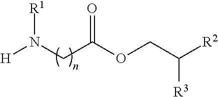

wherein R¹ is chosen from hydrogen, an oxygen atom, and C₁-C₆ alkyl, wherein the C₁-C₆ alkyl may be substituted with carboxylates, hydroxyls, sulfonyls, or sulfonates; n is an integer from 2 to 5 (including 2 and 5); R² is C₅-C₁₂ alkyl; R³ is C₃-C₁₀ alkyl; the terminal nitrogen is optionally further substituted with R⁴, wherein R⁴ is chosen from hydrogen, an oxygen atom, and C₁-C₆ alkyl, wherein the C₁-C₆ alkyl may be substituted with carboxylates, hydroxyls, sulfonyls, or sulfonates; and an optional counterion may be associated with the compound and, if present, the counterion may be 4-methylbenzenesulfonate.

Aspect 2 is the compound of Aspect 1, wherein the compound is 6-((2-butyloctyl)oxy)-6-oxohexan-1-aminium 4-methylbenzenesulfonate, having the following formula:

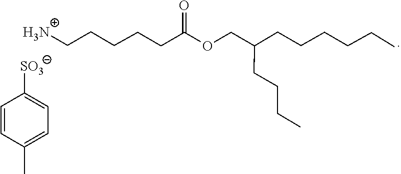

Aspect 3 is the compound of either Aspect 1 or Aspect 2, having a critical micelle concentration (CMC) of about 2.1 mmol in water.

Aspect 4 is the compound of any of Aspects 1-3, having a plateau value of a minimum surface tension of about 27 mN/m.

Aspect 5 is the compound of any of Aspects 1-4, having a surface tension in water equal to or less than 28.5 mN/m at a concentration of 1.0 mmol or greater.

Aspect 6 is the compound of any of Aspects 1-5, having a surface tension in water equal to or less than 30 mN/m at a surface age of 100 ms or greater.

Aspect 7 is a liquid composition comprising: a medium; and a surfactant of the following formula:

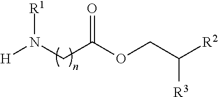

wherein R¹ is chosen from hydrogen, an oxygen atom, and C₁-C₆ alkyl, wherein the C₁-C₆ alkyl may be substituted with carboxylates, hydroxyls, sulfonyls, or sulfonates; n is an integer from 2 to 5 (including 2 and 5); R² is C₅-C₁₂ alkyl; R³ is C₃-C₁₀ alkyl; the terminal nitrogen is optionally further substituted with R⁴, wherein R⁴ is chosen from hydrogen, an oxygen atom, and C₁-C₆ alkyl, wherein the C₁-C₆ alkyl may be substituted with carboxylates, hydroxyls, sulfonyls, or sulfonates; and an optional counterion may be associated with the compound and, if present, the counterion may be 4-methylbenzene sulfonate.

Aspect 8 is the composition of Aspect 7, wherein the medium is water.

1. A compound of the following formula: 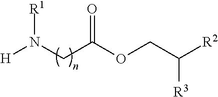
wherein R¹ is chosen from hydrogen, an oxygen atom, and C₁-C₆ alkyl, wherein the C₁-C₆ alkyl may be substituted with carboxylates, hydroxyls, sulfonyls, or sulfonates;
n is an integer from 2 to 5 (including 2 and 5);
R² is C₅-C₁₂ alkyl;
R³ is C₃-C₁₀ alkyl;
the terminal nitrogen is optionally further substituted with R⁴, wherein R⁴ is chosen from hydrogen, an oxygen atom, and C₁-C₆ alkyl, wherein the C₁-C₆ alkyl may be substituted with carboxylates, hydroxyls, sulfonyls, or sulfonates;
and an optional counterion may be associated with the compound and, if present, the counterion may be 4-methylbenzenesulfonate.

2. The compound of claim 1, wherein the compound is 6-((2-butyloctyl)oxy)-6-oxohexan-1-aminium 4-methylbenzenesulfonate, having the following formula:

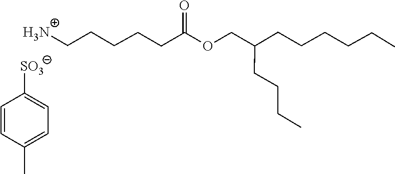

3. The compound of claim 1, having a critical micelle concentration (CMC) of about 2.1 mmol in water.

4. The compound of claim 1, having a plateau value of a minimum surface tension of about 27 mN/m.

5. The compound of claim 1, having a surface tension in water equal to or less than 28.5 mN/m at a concentration of 1.0 mmol or greater.

6. The compound of claim 1, having a surface tension in water equal to or less than 30 mN/m at a surface age of 100 ms or greater.

7. A liquid composition comprising:
a medium;
and a surfactant of the following formula: 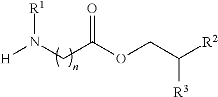
wherein R¹ is chosen from hydrogen, an oxygen atom, and C₁-C₆ alkyl, wherein the C₁-C₆ alkyl may be substituted with carboxylates, hydroxyls, sulfonyls, or sulfonates;
n is an integer from 2 to 5 (including 2 and 5);
R² is C₅-C₁₂ alkyl;
R³ is C₃-C₁₀ alkyl;
the terminal nitrogen is optionally further substituted with R⁴, wherein R⁴ is chosen from hydrogen, an oxygen atom, and C₁-C₆ alkyl, wherein the C₁-C₆ alkyl may be substituted with carboxylates, hydroxyls, sulfonyls, or sulfonates;
and an optional counterion may be associated with the compound and, if present, the counterion may be 4-methylbenzene sulfonate.

8. The composition of claim 7, wherein the medium is water.
